(19) United States
(12) Patent Application Publication
(10) Pub. No.: US 2026/0363575 A1
(43) Pub. Date: Nov. 19, 2026

(54) WEARABLE SYSTEM FOR LONG-TERM ASSESSMENT OF EXPOSURE TO LIFTING HAZARDS

(71) Applicant: University of New Hampshire, Durham, NH (US)

(72) Inventors: Diliang CHEN, Newmarket, NH (US); Femi John OLUGBON, Durham, NH (US); Nozhan GHOREISHI, Dover, NH (US)

(21) Appl. No.: 19/487,775

(22) PCT Filed: May 24, 2024
(86) PCT No.: PCT/US2024/031027

§ 371 Date: Nov. 25, 2025

Related U.S. Application Data
(60) Provisional application No. 63/504,566, filed on May 26, 2023.

(51) Int. Cl. A61B 5/103; A61B 5/00; A61B 5/107; A61B 5/11

(52) U.S. Cl. CPC A61B 5/1036; A61B 5/002; A61B 5/1071; A61B 5/1123; A61B 5/6807; A61B 5/681; A61B 5/7264; A61B 2503/20; A61B 2560/0223; A61B 2560/0257; A61B 2562/0219; A61B 2562/0247; A61B 2562/0252; A61B 2562/046; A61B 2562/164

(57) ABSTRACT

Wearable systems for monitoring parameters associated with load lifting. In one example, a wearable sensor system includes (i) an auto-calibrating first sensor system including a flexible pressure sensor array having a plurality of pressure sensors distributed over a surface area of an insole, and a first IMU, (ii) an auto-calibrating second sensor system configured to be worn on a wrist or hand of a user, and including a second IMU and at least one barometer, and (iii) at least one processor coupled to the first and second sensor systems and configured to implement a first automatic calibration procedure for the flexible pressure sensor array based on first sensor data from the flexible pressure sensor array and the first IMU, and configured to implement a second automatic calibration procedure for the at least one barometer based on the first sensor data and on second sensor data acquired using the second IMU.

### CROSS-REFERENCE TO RELATED APPLICATIONS

This application claims priority to co-pending U.S. Provisional Application No. 63/504,566 filed on May 26, 2023 and titled “UNOBTRUSIVE WEARABLE SYSTEM FOR LONG-TERM ASSESSMENT OF EXPOSURE TO LIFTING HARZARDS IN THE WORKPLACE,” which is hereby incorporated herein by reference in its entirety for all purposes.

### BACKGROUND

Back injuries caused by lifting heavy loads are common and have a profound impact on individual works and society. However, accurate assessment of workers' personal exposure to lifting hazards is largely limited. Lifting risk assessment tools, such as the Revised National Institute for Occupational Safety and Health Lifting Equation (RNLE) and the Washington Industrial Safety and Health Act (WISHA) lifting calculator, are popularly used for assessing exposures to lifting hazards. To perform an exposure assessment using these tools, a professional observer needs to monitor a single worker and manually record the weight of each lifted object, lifting frequency, etc. The time spent observing a worker is often in the range of 1-8 hours per job, depending on the job tasks.

### SUMMARY

Aspects and embodiments are directed to techniques for providing a wearable sensor system that has automatic built-in self-calibration and can be used for long-term assessment of risks associated with load weight, asymmetry angle, load vertical location, load vertical travel distance, load horizontal location, and/or lifting frequency. In some examples, the wearable system has an unobtrusive, user-friendly design, with sensors incorporated into wearable items such as gloves, insoles, and/or wristbands.

As described further below, in one example, a wearable sensor system for lifted load measurements comprises a pair of auto-calibrating insole apparatuses, each including an insole, a flexible pressure sensor array coupled to the insole and including a plurality of pressure sensors distributed over a surface area of the insole, a force plate integrated into a heel region of the insole, and a first inertial measurement unit (IMU) coupled to the insole. The wearable sensor system further comprises at least one sensor package/module configured to be worn on one or both wrist(s) or hand(s) of a user.

These and other aspects are described in more detail below.

### BRIEF DESCRIPTION OF THE DRAWINGS

Various aspects of at least one example are discussed below with reference to the accompanying figures, which are not intended to be drawn to scale. The figures are included to provide an illustration and a further understanding of the various aspects and are incorporated in and constitute a part of this disclosure. However, the figures are not intended as a definition of the limits of any particular example. In the figures, the same or similar components that are illustrated are represented by a like reference numeral. For purposes of clarity, every component may not be labeled in every figure. In the figures:

FIG. 1 is a diagram illustrating components of a monitoring system according to aspects of the present disclosure;

FIG. 2 is a block diagram of one example of a wearable sensor system according to aspects of the present disclosure;

FIG. 3 is a block diagram of another example of a wearable sensor system according to aspects of the present disclosure;

FIG. 4 is a diagram illustrating an example of an insole apparatus including components of the wearable sensor system of FIG. 3, according to aspects of the present disclosure;

FIG. 5 is a graph showing an example of ground reaction force over a person's walking stride;

FIG. 6A is a flow diagram of an example of a calibration process for an insole apparatus according to aspects of the present disclosure;

FIG. 6B is a flow diagram of another example of a calibration process for an insole apparatus according to aspects of the present disclosure;

FIG. 7 is a graph showing various ground reaction force curves according to aspects of the present disclosure;

FIG. 8 is a graph showing pressure sensor force estimates according to aspects of the present disclosure;

FIG. 9 is a graph showing load horizontal location as a function of center of pressure variation, according to aspects of the present disclosure;

FIG. 10 is a diagram illustrating reference postures during walking/standing, according to aspects of the present disclosure;

FIG. 11 is a graph showing vertical location change as a function of air pressure change, according to aspects of the present disclosure;

FIG. 12 is a diagram illustrating an example of a reference position that can be used for a calibration process in accord with aspects of the present disclosure;

FIG. 13 is a flow diagram of one example of a process for identifying reference points during standing, according to aspects of the present disclosure;

FIG. 14 is a flow diagram of one example of a process for identifying reference points during walking, according to aspects of the present disclosure;

FIG. 15 is a diagram illustrating an example of applying machine learning models for activity recognition, according to aspects of the present disclosure; and

FIG. 16 is a diagram showing an example of a lifting process, in accord with aspects of the present disclosure.

### DETAILED DESCRIPTION

Back injuries are leading occupational musculoskeletal disorders (MSDs) that have a profound impact on individual workers and society. For example, low-back pain (LBP) is one of the most prevalent health problems in workplaces. Lifting a heavy load, such as during manual material handling, is a significant cause of low-back pain. Parameters such as Load vertical location (LVL), load weight, asymmetry angle, load vertical travel distance, load horizontal location, and lifting frequency, can all be used for evaluating the risk of LBP. For example, studies have indicated that the tension of the multifidus muscle, a key muscle in the lumbar spine, is significantly affected by the LVL during lifting activities. Additionally, there is a correlation between elevated tension in the back muscles during physical tasks such as lifting, and an increased risk of LBP development. Accurate estimation of one or more of the above-mentioned parameters can provide a valuable tool for assessing the exposure to lifting hazards and helping to reduce the risk of LBP.

However, as noted above, accurate assessment of workers' personal exposure to lifting hazards is largely limited. Furthermore, some methods for measuring the above-mentioned parameters are limited in efficiency, accuracy, and user-friendliness, especially in uncontrolled workplace environments. For example, manual observations are time consuming and costly, and can be prone to subjective errors. Computer-vision-based approaches can track at least some lifting hazard parameters, such as LVL, relatively accurately under certain conditions. However, these methods are limited by environmental conditions, such as light, angle of view, obstructions, etc., which can make it challenging and/or impractical to use these methods in uncontrolled workplace environments.

Wearable systems offer potential to address the above problems and provide methods by which lifting hazard parameters can be measured in uncontrolled workplaces or other environments. For example, wearable monitoring systems may allow for continuous measurement and may be less affected by environmental factors such as lighting and/or obstacles. Some wearable systems can use motion tracking based on measurements from inertial measurement unit (IMU) sensors to track load location and asymmetry angles However, some IMU-based wearable systems suffer from integral drifting errors over time. In addition, installing a high number of IMU sensors (e.g. 17) on different body segments can limit a person's mobility and may hinder adoption in real environments.

Thus, a number of challenges remain with regard to achieving the practical use of wearable systems to accurately measure and monitor lifting risk factors including load weight, asymmetry angle, load vertical location, load vertical travel distance, load horizontal location, and lifting frequency.

Accordingly, to efficiently assess exposures to lifting hazards across a wide range of users in workplace or other environments, examples disclosed herein provide an unobtrusive and auto-calibrating wearable system to automate exposure assessments and that can replace time-consuming observations and manual measurements. Examples of the wearable system disclosed herein provide for long-term assessment of exposure to lifting hazards with minimal assistance from workers, and enable the automated, unconstrained, and widespread monitoring of lifting risk factors such as load weight, load vertical location, load vertical travel distance, load horizontal location, asymmetry angle, lifting frequency, and lifting duration.

As described in more detail below, examples of the wearable system use a small number of sensors for measuring load location and asymmetry angle that can be integrated into commonly worn accessories, such as insoles of shoes, wristbands, and/or gloves. Therefore, users do not need to wear additional items. Accordingly, the wearable system is unobtrusive and can be easily accepted by users. In addition, as described in more detail below, the wearable system can implement automatic calibration, such that it can realize accurate and reliable monitoring throughout the workday, for example, without the need for manual calibration. Examples of the wearable system can provide automatic, continuous, and personalized exposure assessment that has the potential to supply unique data to facilitate better understanding of the relationship between back injuries and improper lifting, help occupational health professionals and employers to apply effective workplace practices and ergonomic solutions to prevent and address back injuries, and improve workers' health.

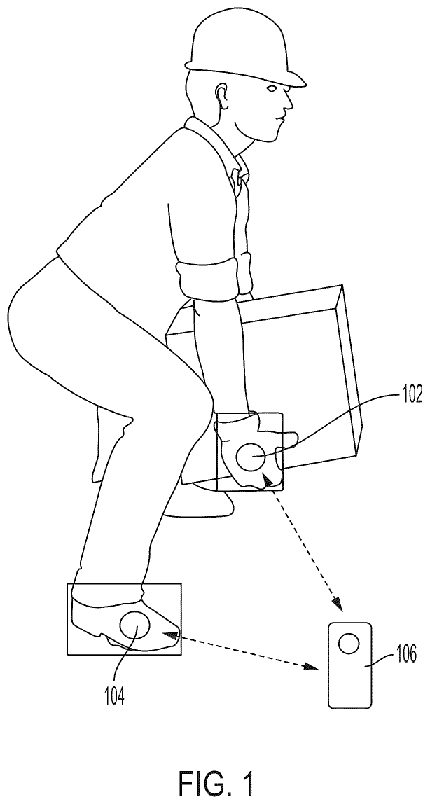

Referring to FIG. 1, according to certain examples, a monitoring system comprises one or more first sensor packages 102 that can be worn on the hand(s) and/or wrist(s) of a user, and one or more second sensor packages 104 that can be integrated into insoles of shoes worn by the user. In some examples, the wearable monitoring system includes a pair of first sensor packages 102 (one worn on each hand/wrist) and a pair of second sensor packages 104 (one worn in each shoe); however, in other examples, the monitoring system may include only one first sensor package 102 and two second sensor packages 104, or only one second sensor package 104 and two first sensor packages 102. Thus, unlike systems that use numerous sensors positioned around the body, examples of the monitoring system disclosed herein can use sensors attached only to insoles and gloves/wrist bands to measure critical parameters for lifting risk assessment. These locations are easily acceptable by users because people are generally used to wearing accessories such as insoles, gloves, and wrist watches. The monitoring system may further include a software application running on a computing device 106 that is in wireless communication with the first and second sensor packages 102, 104, as described further below. Examples of the wearable monitoring system can be used to monitor LVL accurately in uncontrolled environments, such as workplaces.

In some examples, the first sensor package 102 is used to measure air pressure and wrist motion, which may be used for reliable LVL estimation. For example, the LVL can be measured by tracking the vertical location of a user's wrist because the load is lifted with the hands which makes the load close to the wrist. The first sensor package can be used to measure parameters such as asymmetry angle, vertical/horizontal location/distance, lift frequency, and/or lift duration. As described further below, the second sensor package 104 is used for measuring plantar pressure and foot motion. These data can be used to recognize calibration and working activities such as standing, walking, and lifting, for example. As described further below, in some examples, the monitoring system implements an auto-calibration technique referred to herein as known vertical location update (KVLU). Certain KVLU reference points used for auto-calibrating drift in LVL measurements can be identified from standing and walking activities, as described further below. In some examples, the second sensor package 104 can be used to measure load weight, as described further below. The computing device 106 and software application can be used for real-time collection and analysis of the sensor data transmitted via a wireless connection (e.g., BLUETOOTH) from the first sensor package(s) 102 and the second sensor package(s) 104.

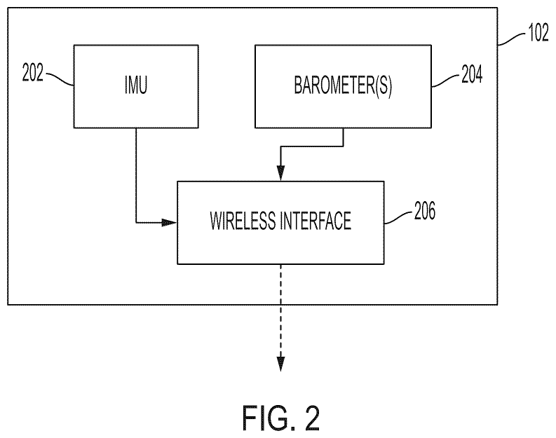

The first sensor package 102 is also referred to herein as a “smart wristband,” although it will be appreciated that the first sensor package may be integrated into a glove or other accessory and is not limited to integration into a wristband. Referring to FIG. 2, in some examples, the first sensor package or smart wristband 102 comprises an inertial measurement unit (IMU) sensor 202 for sensing wrist motion, and a barometer sensor 204 for measuring air pressure. The barometer sensor 204 may include at least one barometer. The smart wristband 102 may further include a wireless communication interface 206 for sensor data collection from the IMU 202 and/or barometer sensor 204 and wireless transmission of the sensor data via a wireless protocol/network, such as BLUETOOTH or WI-FI. In some examples, LVL can be measured by tracking, via the smart wristband 102, the vertical location of a user's wrist during an activity such as lifting, for example, as described further below. In one example, the dimensions of a housing that houses the components of the smart wristband 102 are approximately 40 millimeters (mm) by 24 mm, which may allow the smart wristband to be easily and comfortably worn on the user's wrist(s).

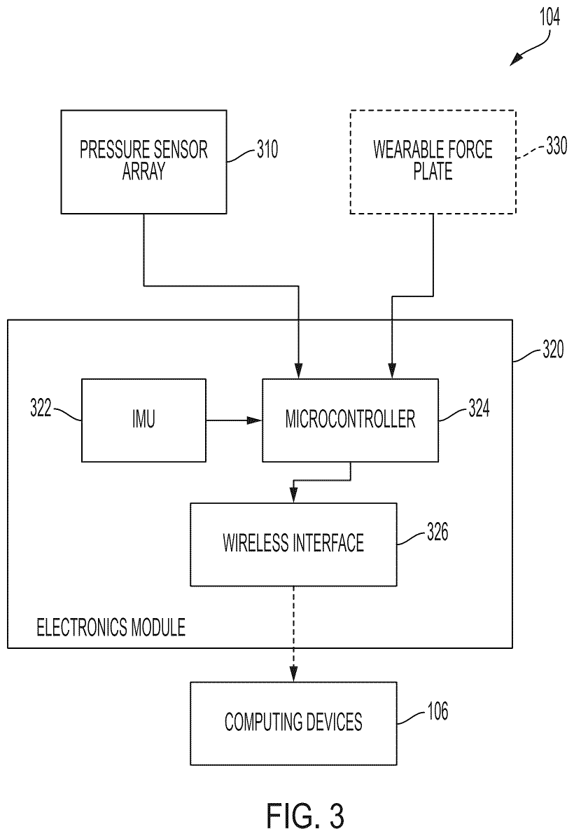

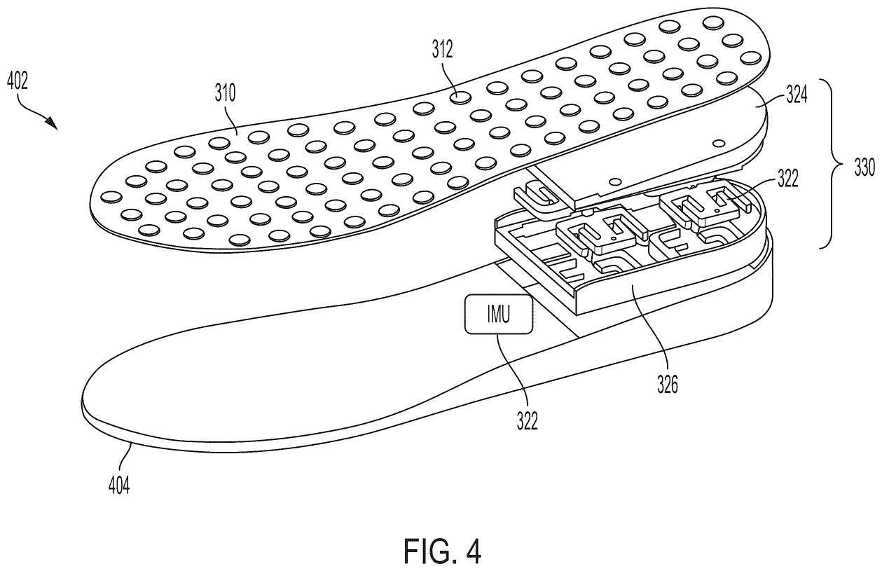

As described above, in some examples, at least some components of the second sensor package 104 can be integrated into an insole worn in a user's shoe. Accordingly, the second sensor package 104 is referred to herein as a “smart insole.” However, as described below, in some instances, at least some components of the second sensor package/smart insole 104 are located separate from an insole that incorporates other components of the second sensor package/smart insole 104. Referring to FIG. 3, in some examples, the second sensor package 104 includes a flexible pressure sensor array 310 for measuring plantar pressure and an electronics module 320. The second sensor package 104 may optionally include a wearable force plate 330 for measuring heel force, as described further below. In some examples, the electronics module 320 includes an IMU 322 for foot motion tracking, and a wireless communication interface 326 for transmitting (e.g., via BLUETOOTH or WI-FI) sensor data to the computing device 106 for further processing. Although the IMU 322 is shown in FIG. 3 as part of the electronics module 200, in other examples, the IMU 322 may be a separate device/module external to the electronics module 320 (e.g., similar to the flexible pressure sensor array 310), as described further below with reference to FIG. 4. In one example, the IMU 322 is a 6-axis IMU that integrates a 3-axis accelerometer with a 3-axis gyroscope. However, in other examples, other IMU configurations can be used. The electronics module 320 may further include a microcontroller 324 that is used to collect sensor data from the pressure sensor array 310 and the IMU 322 (and optionally from the wearable force plate 320) and to send the collected sensor data to the computing device 106 via the wireless communication interface 326. In some examples, the electronics module 320 can be implemented on a printed circuit board. The printed circuit board may have dimensions of approximately 46 mm×46 mm, which makes it easy to be fixed onto a shoe, such as onto shoelaces, for example.

Referring to FIG. 4, there is illustrated an example of a smart insole 402 that may form part of the second sensor package 104 according to certain examples. The smart insole 402 can be used for long-term and accurate measurement of load weight. The components of the smart insole 402 can be incorporated into some examples, the insole apparatus 100 an insole 404 As described above, the smart insole 402 includes the flexible pressure sensor array 310 that includes a plurality of pressure sensors 312. The flexible pressure sensor array 310 can be used for measuring ground force. In some examples, the flexible pressure sensor array 310 may conform generally to the shape of the insole 404, such that the pressure sensors 312 are distributed over the area of the foot when the insole 404 is worn. In other examples, the pressure sensors 312 may be distributed over only a portion of the foot area. In one example, the flexible pressure sensor array 310 includes 96 pressure sensors 312 distributed over the foot area; however, any number of sensors may be used. The flexible pressure sensor array 310 may have a thickness in a range of about 1 millimeter (mm) to 2 mm. In some examples, the flexible pressure sensor array 310 can be trimmable to fit different sizes of foot (or different sizes of the insole 102).

In some examples, the smart insole 402 includes the wearable force plate 330 that is positioned in the heel area of the insole 404. The wearable force plate 330 may supply an absolute force reference for auto-calibration of the flexible pressure sensor array 310, as described in more detail below. In some examples, the wearable force plate 330 is constructed with load cells 332, which are physical transducers that can translate force into an electrical signal. In the illustrated example, the wearable force plate 330 includes a plurality of load cells 332 housed in a housing comprises upper and lower casings 334, 336, respectively, that can be integrated into the heel area of the insole 404. The wearable force plate 330 may be constructed using accurate and stable micro load cells 332. In one example, four micro load cells 332 are placed on four corners of the wearable force plate 330 to ensure that load forces can be stably applied to each load cell during different activities. Micro load cells are frequently used for building body weight scales, which have good accuracy (±0.1%), repeatability (±0.1%), and measurement range (up to 75 kg/load cell). Accordingly, the micro load cells can be used for calibrating the flexible pressure sensor array 410. In addition, micro load cells are thin (2.5 mm) and lightweight (9.7 g), which makes them suitable for building wearable force plates.

In some examples the wearable force plate 330 is integrated into the insole 404 for long-term measurement, and accordingly, the design and construction of the force plate 330 may consider both measurement accuracy and wear comfort. To provide good measurement accuracy, the wearable force plate 330 is configured and positioned in the insole 404 such that all the load forces go through the part of the load cell where its deformation is sensed. The wearable force plate 330 may be rigid. In particular, the upper and lower casings 334, 336 that protect the load cells 332 may be made of a rigid material. To provide wear comfort, examples of the wearable force plate 330 are thin, lightweight, and positioned in the foot area that does not bend. Accordingly, in some examples, the wearable force plate 330 can be integrated into the heel area of the insole 404, as shown in FIG. 4, because the heel area of the human foot generally does not bend during different activities, including walking.

In other examples, however, the smart insole 402 and/or second sensor package 104 omit the wearable force plate 330.

As noted above, in some examples, the IMU 322 may be separate from the electronics module 320. Thus, still referring to FIG. 4, in some examples, the IMU 322 is integrated with the insole 402, while the electronics module 320 may be external to the insole 404. However, in other examples, the IMU 322 is external to the insole (e.g., part of a module attached to shoelaces or another part of a shoe in which the insole 404 is fitted). In other examples, the electronics module 320, including the IMU 322, is integrated with the insole 404. Thus, in some examples, the IMU 322 in FIG. 4 may be replaced with the electronics module 320.

In some examples, the IMU 322, and optionally the wearable force place 330, are used to automatically calibrate the flexible pressure sensor array 310 during load-weight measurements, as described further below. Examples of the insole 402 can provide long-term accuracy for plantar pressure measurements. Various examples may further provide a personalized activity recognition method to increase the accuracy and reliability of recognizing the lifting activity in uncontrolled workplace settings. Further examples may identify frequently used walking postures to be used as references for automatically correcting drifting errors associated with the IMUs and barometers of the first sensor package 102.

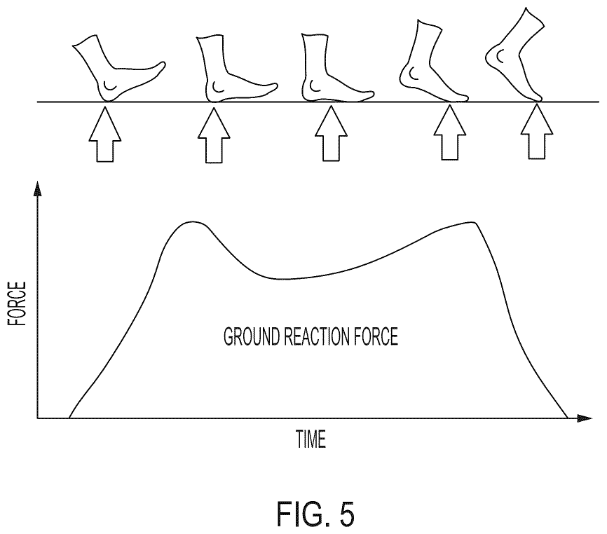

According to certain embodiments, load weight can be measured with a calibrated flexible pressure sensor array 310. Based on an assumption that all the weight (body weight and load weight) will be applied on both feet during the lifting activity, flexible pressure sensor arrays under foot can be used to measure the ground reaction force (GRF), which can be used to estimate the total weight. Referring to FIG. 5, GRF is the force exerted by the ground on a body in contact with the ground. GRF is the force that propels the human body, enabling various activities, and it reflects information about locomotion, gait, and injury risk associated with human movement. For example, FIG. 5 is a graph illustrating GRF at different stages of a person's walking gait. As a person walks, different areas of the foot are in contact with the ground over time, as illustrated in FIG. 5. Accordingly, a wearable pressure sensor, such as the flexible pressure sensor array 310 integrated into the insole 404, for example, can provide GRF measurements.

According to certain examples, a method for calibrating the flexible pressure sensor array 310 includes estimating the GRF pattern during an activity, such as straight walking, for example. In some examples, a machine-learning model using sensor data from the IMU 322 and optionally the flexible pressure sensor array 310 is configured to estimate the complete GRF during walking. In some examples, the wearable force plate 330 introduced into the heel area of the insole 404 used to measure the GRF in the heel area and to supply reference force measurements that can used as part of the calibration process described below. However, as noted above, in some examples, the smart insole 104/402 omits the wearable force plate 330, and information such as know body weight can be used instead of the reference force measurements, as described further below. To realize an accurate calibration of each individual sensor 112 of the flexible pressure sensor array 110, a processing technique is applied that includes three steps: estimating an average regression curve for all sensors, re-estimating the true pressure on each sensor during walking, and obtaining the true regression curve of each sensor. This approach eliminates the limitations of manual calibration, making smart insole technologies more accessible for everyday use.

According to certain examples, to calibrate the flexible pressure sensor array 110, measurements or estimates of the force over a portion of the foot area or the whole foot area are used. In some examples, foot motion is used to estimate the total pressure based on the close relationship between foot motion and the pressure under foot (as illustrated in FIG. 5, for example). According to certain examples, the calibration process is performed during walking. Because GRF is the force to enable different activities, its pattern is closely related to different motions. It has been demonstrated that the GRF pattern during complex activities, such as jumping, can be estimated using IMU sensors on the pelvis and both feet. Accordingly, in examples of the methodology disclosed herein, the IMU sensor 322 on the foot (e.g., in the insole 404) are used to estimate the GRF pattern during walking. Walking is a frequently used activity. Furthermore, the pattern of GRF across the foot during walking motion is relatively simple and there is always a foot in contact with the ground. These attributes make walking a good activity to use for calibration of the flexible pressure sensor array 310. However, it will be appreciated, given the benefit of this disclosure, that although examples below describe performing the calibration during walking, in other examples, the calibration processes can be performed during activities other than walking (such as, but not limited to, running, jumping, standing, etc.).

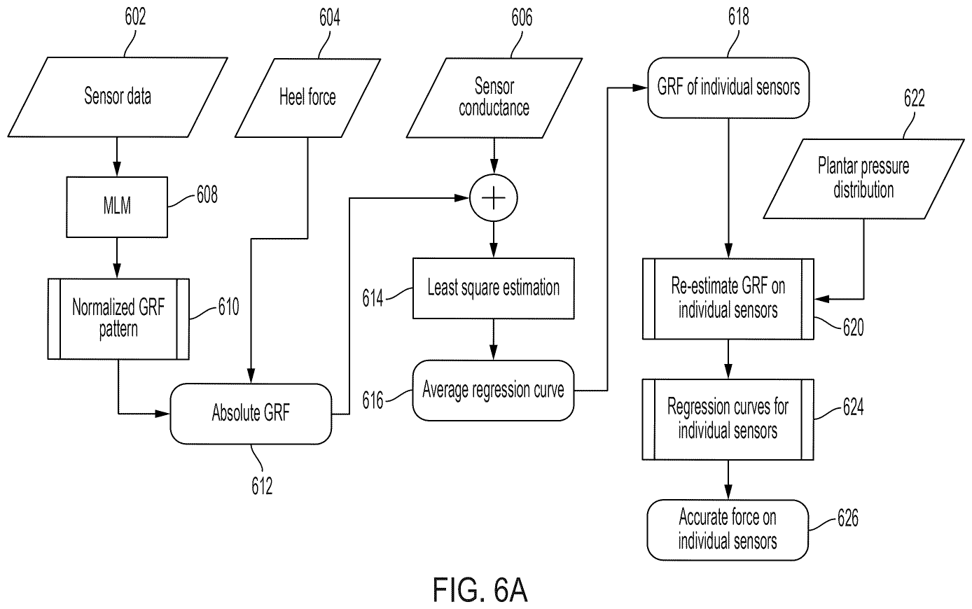

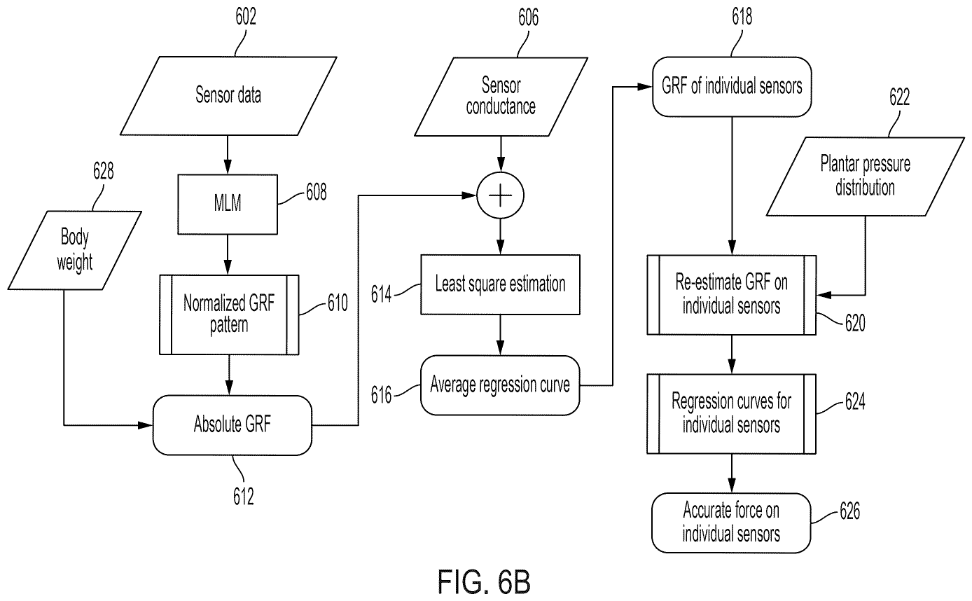

Examples of an automatic calibration process for the flexible pressure sensor array 310 are described below with reference to FIGS. 6A and 6B. Aspects of the calibration methods disclosed herein may be implemented on the microcontroller 324 and/or the computing device 106, such as a smartphone or tablet, for example.

Referring to FIG. 6A, a calibration process may use sensor data collected from various components of the smart insole 104. According to certain examples, a machine learning model (MLM) can be trained to predict the GRF pattern during walking based on sensor data 602 and correlations between GRF and foot motion during walking. The sensor data 602 may include information, such as acceleration, angle, and angular velocity, that can be measured by the IMU 322 during walking, for example. The sensor data 602 may further include center of pressed sensors (COPP) data that can be provided by the flexible pressure sensor array 310. Accordingly, at operation 608, the machine learning model may produce a normalized GRF pattern 610 that corresponds to whole foot motion during walking. In some examples, the machine learning model can be programmed into one or more processors (e.g., the microprocessor 324) that can be integrated with the insole 404 or positioned external to the insole 404, and is communicatively coupled to the various sensors in the insole via one or more wired or wireless communication link(s). In some examples, the machine learning model can be implemented in an application running on the computing device 106. In such instances, the sensor data 602 may be communicated to the computing device 106 via the wireless interface 326, as described above.

According to certain examples, the machine learning model is implemented as an artificial neural network (ANN). In some example, the machine learning model is implemented as a long short-term memory (LSTM) machine learning model that can be trained to estimate GRF patterns, for example, during walking or other activities. In some examples, the LSTM model can be trained using data acquired from the IMU 322 and COPP signals from the flexible pressure sensor array 310. Although the COPP signals are obtained from the flexible pressure sensor array 310, the COPP data may remain unaffected by the drifting errors that may be inherent to the flexible pressure sensor array by treating individual pressure sensors 312 in the array as pressed switches to obtain the COPP signal.

According to certain examples, the wearable force plate 330 can supply an absolute reference force to calibrate the part of flexible pressure sensor array 310 at the heel area in real-time. During the heel contact phase of walking, the true GRF can be measured by the wearable force plate 330. Thus, during walking, the wearable force plate 330 may measure heel force to produce sensor data 604. For example, the sensor data 604 from the wearable force plate 330 may provide an absolute reference GRF pattern for the heel region of the foot corresponding to the measured heel force during walking. According to certain examples, a Multiplication Factor (MF1) can be obtained to match the GRF measured by the flexible pressure sensor array 310 (heel area) with the absolute reference force measured by the wearable force plate 330. However, the MF1 cannot be applied directly to calibrate the flexible pressure sensor array at the forefoot area because the factors (e.g. bending, pressure, temperature, and humidity) that cause flexible pressure sensor array 310 drifting are different in the heel and the forefoot areas, which will lead to different drift errors. Accordingly, examples provide processes to calibrate the midfoot and forefoot sensors, as described further below.

Still referring to FIG. 6A, in some examples, a combination of the normalized GRF pattern 610 obtained from the machine learning model (e.g., using a trained LSTM as described above) at operation 608 and reference heel force measurements (sensor data 604) obtained from the wearable force plate 330 can be used to estimate an absolute GRF 612 during walking. For example, the multiplication factor MF1 can be used to match the normalized GRF pattern to the absolute GRF. For example, the absolute GRF 612 may be estimated by using the reference heel force measurements from the wearable force plate 120 to convert the normalized GRF pattern 610 produced by the machine learning model at operation 608 into units of force (e.g., Newtons).

Using the estimated GRF pattern from the machine learning model (trained using any combination of sensor data as described above) in combination with reference force measurements from the wearable force plate 330 to produce the absolute estimated GRF 612 may provide a high accuracy method by which to calibrate the flexible pressure sensor array 310 according to processes described herein. However, in other examples, automatic calibration of the flexible pressure sensor array 310 may be accomplished without using the wearable force plate 330. For example, referring to FIG. 6B, in some examples, the absolute estimated GRF 612 can be obtained using body weight information 628 (e.g., a known body weight of an individual wearing the smart insole 104) to convert the normalized GRF pattern 610 into units of force. In some examples, omitting the wearable force plate 330 may increase cost-efficiency and comfort associated with the smart insole 104.

Thus, in some examples, the smart insole or second sensor package 104 includes the flexible pressure sensor array 310, the IMU 322, and the wearable force plate 330. An automatic calibration process for the flexible pressure sensor array 110 may include training an LSTM machine learning model with COPP and IMU sensor data, and using the trained LSTM machine learning model to estimate the normalized GRF pattern 610. The calibration process may further include using reference force measurements from the wearable force plate 330 to estimate the GRF in Newtons (e.g., produce the absolute estimated GRF 612 as described above). This approach may provide a very high accuracy calibration, in combination with the remaining process steps described below.

In other examples, the smart insole 104 includes the flexible pressure sensor array 310, the IMU 322 (omitting the wearable force plate 330). In some such examples, an automatic calibration process for the flexible pressure sensor array 310 may include training an LSTM machine learning model with COPP and IMU sensor data, and using the trained LSTM machine learning model to estimate the normalized GRF pattern 610. The calibration process may further include using known body weight information to estimate the GRF in Newtons (e.g., produce the absolute estimated GRF 612 as described above). This approach (in combination with process steps described below) may achieve calibration of the flexible pressure sensor array 310 with good accuracy.

In some examples, the estimated absolute GRF 612 may be an estimate of the GRF for the whole foot during walking. However, to calibrate the flexible pressure sensor array 310, estimates of the GRF for regions of the insole 404 corresponding to different regions of the foot (e.g., heel, mid-foot, forefoot), estimates of the GRF measured by individual sensors in the array, and/or estimates of the GRF during activities other than walking, may be needed. Accordingly, in certain examples, sensor measurements are obtained from the flexible pressure sensor array 310 to produce sensor data 606 that may be used to produce estimated GRF for regions of the foot area and/or individual sensors 312. In one example, the sensor measurements are sensor conductance measurements; however, in other configurations, sensor resistance or other measurements may be used.

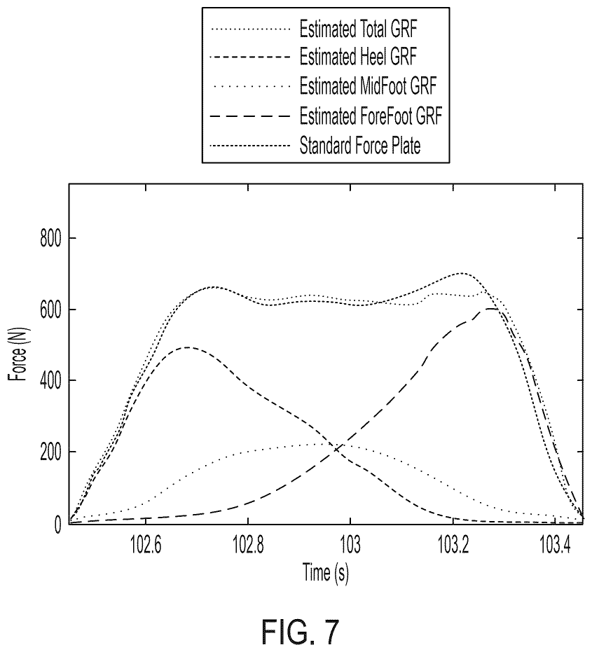

Considering the foot structure, the foot can be separated into three areas: heel, Mid-foot, and forefoot. Referring to FIGS. 6A and 6B, based on an assumption that only sensors 312 in the same foot area (e.g., heel, mid-foot, forefoot) have the same average regression curve, in one example, three different regression curves can be estimated for sensors 312 in the heel, mid-foot, and forefoot areas, respectively. At operation 614, a least squares estimation, or other estimation technique, may be applied to a combination of the sensor data 606 from the flexible pressure sensor array 310 (e.g., sensor conductance measurements) and at least a corresponding portion of the estimated absolute GRF 612 to produce, at operation 616, an average regression curve for different foot regions (e.g., heel, mid-foot, forefoot). For example, sensor measurements from the heel region of the insole 404/flexible pressure sensor array 310 in combination with the estimated absolute GRF 612 for the heel region of the can be used to produce an average regression curve of the heel sensors. Similarly, sensor measurements from the individual sensors 312 in other foot regions (e.g., forefoot/toe, mid-foot, etc.) and corresponding portions of the estimated absolute GRF 612 can be used to produce average regression curves for those groups of sensors. Based on the average regression curves of different foot regions, estimated GRF corresponding to the different foot regions can be produced during all (or at least several) activities, rather than only walking. The summation of all the regional GRF is the complete GRF under foot, as shown in FIG. 7, for example. The estimated regional GRF curves are produced based on the average regression curves of different regions, determined at operation 616, and their summation is the complete total GRF under foot.

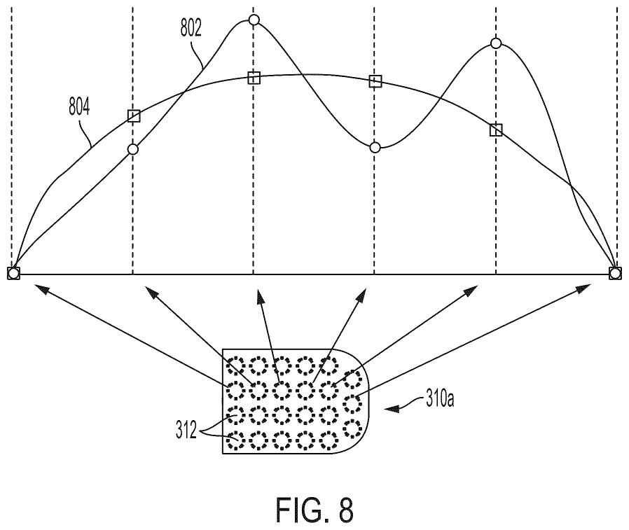

At operation 618, an estimate of the GRF at some or all individual sensors 312 in the flexible pressure sensor array 310 can be obtained. This information can then be used to calibrate the flexible pressure sensor array 310. According to certain examples, information about the geometry of the foot can be used to adjust the average regression curve obtained at operation 616 for individual sensors 312 within the flexible pressure sensor array 310. For example, known information about the plantar pressure in the heel area, and/or other areas of the foot (e.g., plantar pressure distribution information 622), can be used to adjust the regression curve values for individual sensors 312. For example, FIG. 8 illustrates a curve 802 representing estimated sensor pressure for a heel region of the foot based on an average regression curve produced at operation 616. Referring to FIGS. 6 and 8, at operation 620, GRF values for individual sensors 312 in the corresponding portion 310a of the insole 404 can be adjusted to produce a more accurate estimate of the GRF at the individual sensors. FIG. 8 further illustrates a curve 804 showing re-estimated sensor pressure (force) measurements for sensors 312 in the portion 310a of the insole 404. These force estimates can then be used to calibrate the flexible pressure sensor array 310. For example, as shown in FIGS. 6A and 6B, at operation 624, regression curves for individual sensors 312 can be produced based on the re-estimated GRF values at the individual sensors (obtained at operation 622). Using the individual regression curves obtained at operation 624, an accurate estimate of the force at each individual sensor can be obtained at operation 626.

As described above, examples of the monitoring system disclosed herein can be used to measure a variety of parameters associated with lifting or other tasks that can then be used to assess a person's risk of injury or other health concerns related to such tasks. For example, such parameters may include load weight, asymmetry angle, load vertical location, load vertical travel distance, and/or load horizontal location.

According to certain examples, the measured GRF under foot is used to estimate the weight. For static activities like standing, the measured GRF is the weight. During dynamic activities, the measured force under foot will fluctuate around the true weight. Different methods can be used for estimating forces during different dynamic activities: cyclic activities and non-cyclic activities. For cyclic activities like walking, the mean value of the GRF during one gait cycle can be used to estimate the weight, based on the Newton's Laws of Motion; For non-cyclic activities, a low-pass filter can be used to remove the spike noises and estimate the true value. Body weight can be measured during neutral walking activities, for example, and load weight can be measured by subtracting the body weight from the total weight.

According to certain examples, load horizontal location can be measured with the calibrated flexible pressure sensor array 310. To reach to a load with a higher horizontal location, the worker needs to lean forward, which causes the center of gravity (CoG) of the whole body move forward. It has been shown that the center of pressure (CoP) measured under foot is the vertical projection of the CoG of the body and any load on the hands. Accordingly, the load horizontal location can be detected by the CoP.

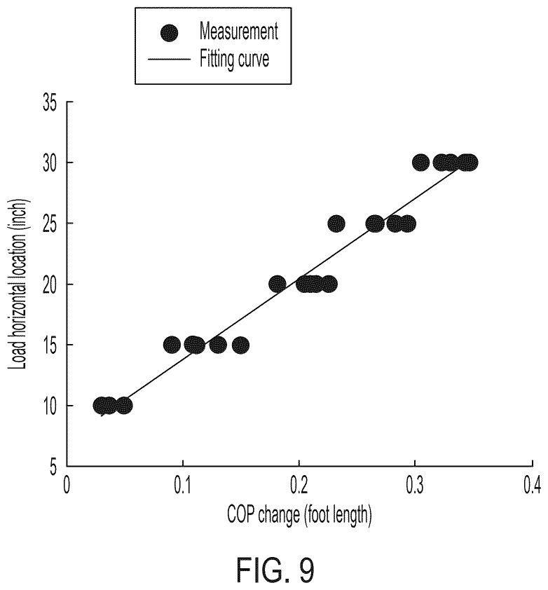

FIG. 9 is a graph showing the relationship between the CoP change and the load horizontal location. The data presented in FIG. 9 was obtained from measurements of CoP change made at five different load horizontal locations (10, 15, 20, 25, 30 inches). As shown in FIG. 9, there is a linear relationship (R2=0.97) between the COP change and load horizontal location. The mean±Standard Deviation accuracy of load horizontal location estimated with the CoP change was 0.99±0.77 inch.

According to certain examples, the CoP during normal standing is used as a reference CoP for calculating the CoP change. The reference CoP may be automatically measured during standing, as described further below. In some examples, the median value of the CoP during standing can be used as the reference to avoid the influence of spike noises.

According to certain examples, asymmetry angle can be measured using the IMUs 202 in the smart wristbands 102 and the IMUs 322 in the smart insoles 104. Asymmetry angle may measure how far the load is displaced from the mid-sagittal plane of the worker's body at the beginning or ending of the lift in degrees. The mid-sagittal plane is an anatomical plane that separates the body into right and left sides, where forward and backward motions like walking will occur. The feet and the torso are generally tightly coupled during gait. Accordingly, in certain examples, the average of the angle of both feet can be used as the reference to calculate the asymmetry angle. Examples of the smart insole 104 may measure turning angle with good accuracy (e.g., approximately 1.93 degrees).

For manual lifting tasks with both hands, the worker will need to use both hands to reach to the direction of the load before lifting, then grasp and lift it up, then transfer the load to the direction of the destination, and finally release the hands. Since the hands are involved in the whole lifting process, the load angle can be tracked with IMUs on the wrists (e.g., the IMU 202). Therefore, the asymmetry angle can be calculated measuring the angular differences between the feet and hands. However, a gyroscope (one sensor in the IMU) may have integration drift over time, which can make it challenging to obtain accurate angular measurements during long-term measurement. According to certain examples, zero-asymmetry reference postures are identified and used to calibrate the asymmetry angles measured by the IMUs on the wrists and feet and remove the integration drift, as described further below.

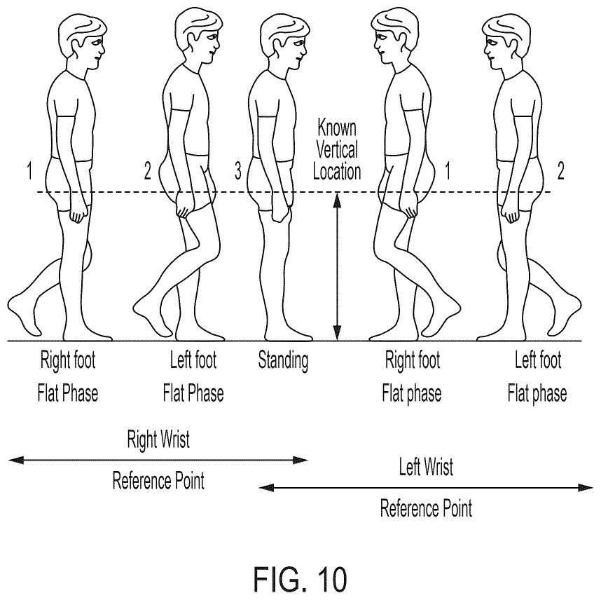

For example, as shown in FIG. 10, in one example, a first zero-asymmetry reference posture is walking with hands on the side of the body. During walking, arms will swing forward and backward rhythmically. Since during walking, the whole body, including the feet, torso, and hands are moving along the mid-sagittal plane, the asymmetry angle=total hands angle/2−total feet angle/2=0.

In some examples, the smart insole 104 can be used to identify daily activities, such as walking, running, stair ascending, and stair descending, for example. Therefore, examples of the smart insole 104 are able to detect a designated activity, such as straight walking, for example, for calibrating the flexible pressure sensor array 310 automatically, as described above.

According to certain examples, the flexible pressure sensor array 310 and the accelerometer (in the IMU 322 and/or IMU 202) can detect the walking and the hand posture reliably. As discussed above, examples of the insole 102 can recognize daily activities, such as walking, running, stair ascending, and stair descending. Through the IMU sensor on the wrists (e.g., in sensor module 104), it can be determined whether the hands are moving on the side of the body rhythmically. Therefore, examples of the wearable system disclosed herein are able to detect zero-asymmetry walking activity for calibrating various system components automatically.

Additionally, a second zero-asymmetry reference posture is the symmetry lifting. When the lifting is symmetric, both the body center of mass (COM) and load COM will be in the mid-sagittal plane, and the force on each supporting foot will be balanced. Otherwise, the CoP will move to the side of the CoM. With the auto-calibrated flexible pressure sensor array 310, as described above, the measured CoP can be used to identify symmetry lifting with parallel and staggered feet lifting. Using these two zero-asymmetry reference postures, lifting asymmetry angles can be measured reliably.

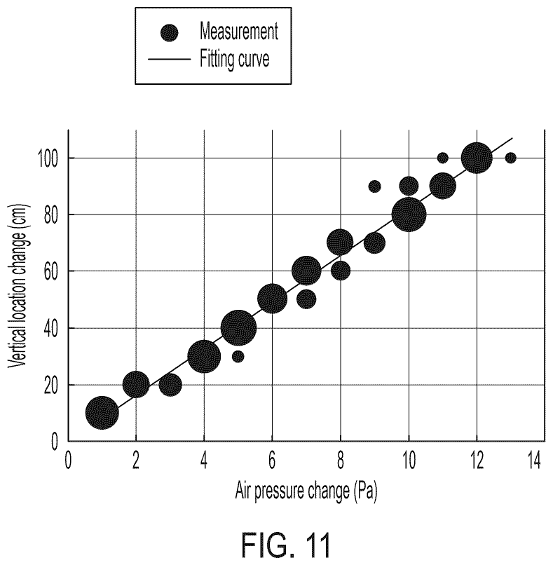

As described above, load vertical location (LVL) can be measured by monitoring the vertical height of a person's wrist because the load is lifted with hands, which makes the load near to the wrist. According to certain examples, LVL can be measured using the barometer sensor 204 included in the first sensor package 102. Due to the highly dynamic nature of hand activities, conventional IMU-based hand motion capture suffers from serious drift and instability problems. The barometer sensor 204 offers an alternative method for tracking vertical location changes. In some examples, the barometer sensor 204 can be used for estimating wrist vertical travel distance by measuring the air pressure changes during short-term lifting events. For example, FIG. 11 is a graph showing the relationship between the air pressure change and the vertical location change. The data presented in FIG. 11 was obtained from ten measurements performed for each vertical location change from 0 cm-10 cm to 0 cm-100 cm, for a collection of a total of 100 sample data points. As shown in FIG. 11, the change in the air pressure showed a linear relationship (R2=0.98) with the change of vertical locations. The mean #standard deviation accuracy of vertical location change estimated with the air pressure change was 3.07±2.55 cm. In FIG. 11, the size of the red point indicates the number of corresponding samples.

However, in some instances, measurements from the barometer sensor 204 cannot be directly used to provide a reliable estimate of LVL over a long time (e.g., 8 hours or more) because of the environment-induced drifting errors. To correct the barometer drifting problem over time, techniques are disclosed herein for providing an auto-calibration method. According to some examples, a Known Vertical Location Update (KVLU) calibration method involves synergistically fusing human gait, posture, and anthropometry data. This calibration method may effectively mitigate drift errors in LVL measurement by updating the estimated wrist vertical location with a known value at recognized calibration postures by the wearable system, as described further below.

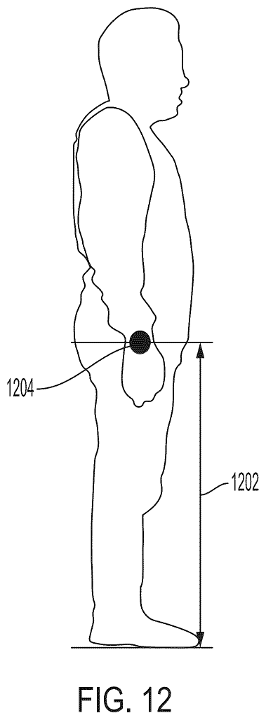

Referring again to FIG. 10, according to certain examples, the known vertical location used in the KVLU calibration process is the distance from the wrist to the ground when the foot(s) are flat, leg(s) are straight, and the wrist is positioned vertically. Since the length of different body segments can be estimated based on the known body height, the known vertical location at the KVLU reference points can also be estimated with the known body height. Thus, referring to FIG. 12, based on a known vertical height 1202, during an identified reference activity, such as normal standing as shown in FIG. 12, for example, a KVLU reference point 1204 can be determined.

Referring again to FIG. 10, the KVLU reference points with the known vertical location may identified in three postures: (1) standing with wrist(s) in the vertical posture; (2) the foot flat phase of the right foot during walking with wrist(s) in a vertical posture; (3) the foot flat phase of the left foot during walking with wrist(s) in a vertical posture. In some examples, in addition to the flat foot phase of walking, the mid-stance phase may be used as well. Since standing and walking are among the most common activities performed by workers, the identified KVLU reference points can be used to frequently calibrate LVL measurement errors and address the issue of barometer drift.

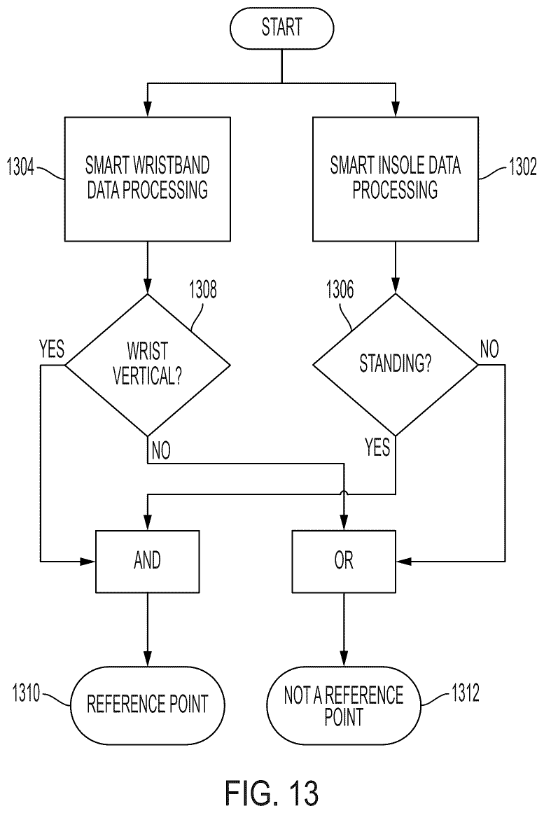

Referring to FIG. 13, there is illustrated a flow diagram for an example of a method of detecting KVLU reference points during standing, according to certain aspects. The method can be performed by at least one processor executing one or more sequences of instructions. The at least one processor may be part of the computing device 106 and/or the microcontroller 324, for example. In the example of FIG. 13, the KVLU reference point 1204 can be identified as the location of the smart wristband 102 when the user is standing normally with the wrist vertically along the body side, as shown in FIG. 12. At operation 1302, standing posture can be accurately recognized with the smart insole 104. Thus, based on processing sensor data from the smart insole 104 at operation 1302, a determination can be made at operation 1306 as to whether or not the user is in a standing posture/position. At operation 1304, the IMU 202 in the smart wristband 102 is used to determine whether the wrist is in a vertical posture. Determined by the placement of the IMU 202 in the smart wristband 102, the measured pitch angle of the wrist increases as the wrist gets closer to the vertical position and decreases as the wrist posture deviates from it. Typically, during normal standing with wrists resting at the sides, the wrists may not be perfectly vertical. Therefore, an angular threshold can be used to determine if the wrists are in a suitable “vertical” posture.

The wrist pitch angle at the KVLU reference points during normal standing may have slight differences among different normal standing postures and normal standing postures performed by different users. It can be assumed that the distribution of the wrist pitch angle at the KVLU reference points during normal standing fits a Gaussian distribution. According to the empirical rule of the Gaussian distribution, nearly all the data (e.g., 99.73%) would lie within three standard deviations of the mean. Therefore, to determine whether the wrist is in a suitable vertical posture, a threshold for the wrist pitch angle can be calculated with Equation 1 as follows:

$${Threshold}_{angle} = \frac{1}{{n}_{angle}} {\sum }_{k = 1}^{{n}_{angle}} ( {angle}_{k} - 3 * {σ}_{angle} ) \qquad ( 1 )$$

In Equation (1), n_{angle} indicates the number of wrist angle samples collected at the KVLU reference point during standing, and σ_{angle} is the standard deviation of those wrist angle samples.

Referring again to FIG. 13, when the measured wrist angle, measured at operation 1304, is larger than the Threshold_{angle}, the wrist is recognized, at operation 1308, as being in a “vertical” posture. If, at operation 1308, the wrist is determined to be in a vertical posture, and at operation 1306, the user is determined to be in a standing position, then at operation 1310, the measured wrist location can be identified as a KVLU reference point. On the other hand, if either condition is not met (e.g., at operation 1308 the wrist is not determined to be in a vertical posture, or at operation 1306, the user is determined not to be in a standing position), then, at operation 1312, the measured wrist position is not identified as a KVLU reference point.

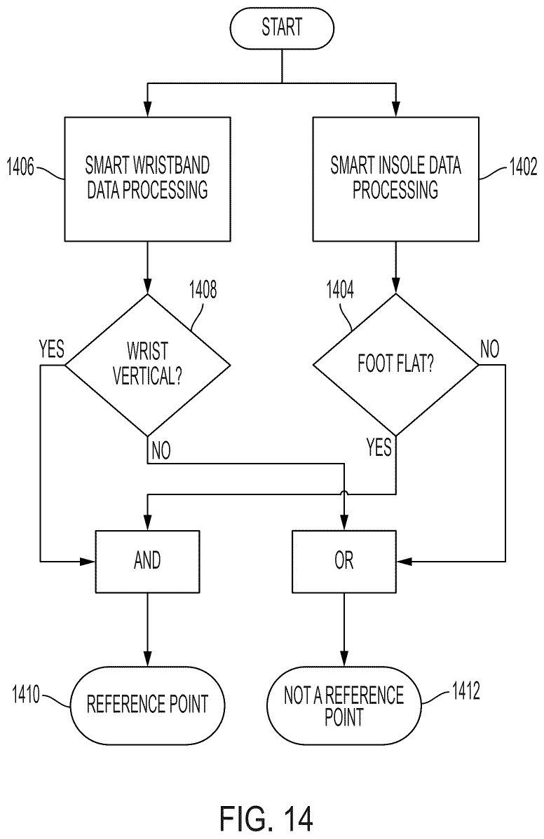

FIG. 14 illustrates a flow diagram of an example of a process for identifying KVLU reference points during walking. The method can be performed by at least one processor executing one or more sequences of instructions. The at least one processor may be part of the computing device 106 and/or the microcontroller 324, for example. In the example of FIG. 14, the KVLU reference points 1204 are identified when the wrist is in a vertical posture and the foot is in the foot flat phase of the walking activity, as shown in FIG. 10, for example. However, as described above, in other examples, the mid-stance phase can be used instead or in addition. Walking can be accurately recognized using the smart insole 104. Since both the heel and forefoot will be in contact with the ground during the foot flat phase, the flexible pressure sensor array 310 in the smart insole 104 can be used to recognize the foot flat phase by detecting, at operation 1402, if the heel and forefoot areas are actively pressed. To accurately determine if the heel or the forefoot area was pressed, dynamic thresholds may be calculated using the following equations 2 and 3.

$${Threshold}_{heel} = \frac{1}{{n}_{swing}} {\sum }_{k = 1}^{{n}_{swing}} ( {HeelGRF}_{k} + 3 * {σ}_{heel} ) \qquad ( 2 )$$

$${Threshold}_{fore} = \frac{1}{{n}_{swing}} {\sum }_{k = 1}^{{n}_{swing}} ( {ForeGRF}_{k} + 3 * {σ}_{fore} ) \qquad ( 3 )$$

These thresholds, Thresholdₕₑₑₗ and Threshold_{fore}, indicate the dynamic thresholds used to determine if the heel area and the forefoot area are pressed, respectively. In Equations (2) and (3), n_{swing} indicates the number of samples during the swing phase when the foot is in the air and the flexible pressure sensor array 310 is not pressed. The HeelGRF and ForeGRF indicate the ground reaction force (GRF) measured by the pressure sensors 312 in the heel area and the forefoot area, respectively, and σₕₑₑₗ and σ_{fore} indicate the standard deviation of HeelGRF and ForeGRF during the swing phase.

In some instances, although the flexible pressure sensor array 310 is not actively pressed during the swing phase, small contact forces exist and the measured HeelGRF and ForeGRF are nonzero. The distribution of the HeelGRF and ForeGRF during the swing phase may fit Gaussian distributions. Thus, if at operation 1404, based on the processing and calculations performed at operation 1402, the measured HeelGRF and ForeGRF are higher than the threshold values, Thresholdₕₑₑₗ and Threshold_{fore}, respectively, the foot flat phase is recognized.

At operation 1406, the smart wristband 102 can be used to determine wrist position. Similar to the process of recognizing the wrist vertical posture during standing (operation 1308), only when the wrist pitch angle (measured at operation 1406) is higher than the Threshold_{angle}, is the wrist posture recognized, at operation 1408, as being in a suitable “vertical” posture. However, during the foot flat phase, the wrist angle can be changed continuously in a range and there could be many candidate samples. Accordingly, in some examples, to identify a suitable candidate sample as the KVLU reference point at operation 1410, the first and last sample above the Threshold_{angle} are selected and only the one closest to the middle of the foot flat phase is used as the KVLU reference point for that gait cycle. This is because the supporting leg is generally more straight near the middle of the foot-flat phase. For selection of a KVLU reference point, a determination is also made at operation 1404 that the user is in the flat foot phase of walking. If the user is not in the flat foot phase of walking or the wrist is determined (at operation 1408) not to be in a vertical posture, at operation 1412, the measured wrist position is not used as a KVLU reference point.

According to certain examples, after the identification of KVLU reference point(s) 1204, the vertical location of the identified KVLU reference point(s) can be estimated, such that the KVLU reference point(s) can be for calibrating the LVL measurement. According to anthropometry, there exists a ratio between the length of different body segments and the body height. Furthermore, the ratio between a specific body segment and the body height is similar across users. Therefore, the vertical location of the wrist at the KVLU reference points when body segments such as legs, trunk, and arms are straight, can be estimated with the known body height and a known ratio. In some examples, the wrist-to-body height ratio can be calculated based on data collected from a group of test subjects. For example, the mean or average of measured wrist-to-body height ratios from the group of test subjects can be used to calculate an estimated generalized wrist-to-body height ratio. This generalized (or averaged) ratio can then be used to estimate the wrist vertical location at the KVLU reference point (e.g., the vertical height of the KVLU reference point 1204) by multiplying the wrist-to-height ration by the known body height of a particular user.

With the known vertical height of the KVLU reference point 1204, the drift errors in LVL measurement can be auto-calibrated. LVL includes two parts: the vertical height at the KVLU reference point and the vertical travel distance relative to the KVLU reference point during lifting. Since the barometer reading changes between the detected KVLU reference points and the lifting points can be measured, the load/wrist vertical travel distance relative to the KVLU reference point can be calculated by applying a linear regression model to the measured barometer reading changes. The LVL then can be calculated by adding the vertical height of the KVLU reference point and the wrist vertical travel distance.

The load vertical travel distance and the corresponding changes in the air pressure measured with the barometer sensor 204 have a linear relationship. Therefore, with the KVLU reference point as a reference, the relationship between the load vertical travel distance and the air pressure changes can be described by Equation (4):

LVL−LVL_{KLVU}=a*(P−P_{KVLU})+b (4)

In Equation (4), LVL and LVL_{KVLU} indicate the true LVL and the known vertical location at the KVLU reference points, respectively; P and P_{KVLU} indicate the barometer-measured air pressure and the air pressure at the KVLU reference points, respectively; and a and b indicate parameters determined by the linear relationship between the load vertical travel distance and the corresponding changes in the measured air pressure.

After transformation, Equation (5) can be used to estimate LVL in real-time. Since environmental-induced drifting errors mainly influence P_{KVLU}, Equation (5) can be updated with the latest P_{KVLU} whenever KVLU reference points are identified. In addition, the drifting errors between KVLU reference points can be estimated based on a linear drifting model. Therefore, the LVL measurement between two KVLU reference points can be further calibrated by removing the estimated linear drift errors.

LVL=a*(P−P_{KVLU})+LVL_{KVLU}+b (5)

According to certain examples, during walking, the KVLU reference points can be determined by the combination of different feet and wrists, including right foot and right wrist (RF-RW), right foot and left wrist (RF-LW), left foot and right wrist (LF-RW), and left foot and left wrist (LF-LW). To calibrate the LVL measurement of one smart wristband 102, two KVLU reference points can be used. For example, the KVLU reference points determined by RF-RW and LF-RW can be used to calibrate the LVL measured by a smart wristband 102 worn on a user's right wrist. Similarly, the KVLU reference points determined by RF-LW and LF-LW can be used to calibrate the LVL measured by a smart wristband 102 worn on the user's left wrist. Experimental results have shown that, when considering both reference points for each wrist, nearly 100% of walking steps can produce qualified KVLU reference points for the smart wristbands 102. This result indicates that KVLU reference points can be frequently identified during walking for LVL calibration. Further, experimental results have shown that calibrated smart wristbands 102 (calibrated using the KVLU methods described above) can be used to produce accurate LVL measurements.

As discussed above, certain examples provide for optimization of the measurement accuracy of the load location and asymmetry angle with personalized activity recognition and zero-reference posture identification. Activities related to manual material handling, including working activities such as lifting, holding, carrying, pushing, and pulling, for example, and daily living activities such as standing and walking, for example. Some of the daily living activities and working activities are similar, making it challenging to discriminate these activities. For example, lifting and holding at the waist level can be similar to standing. Further, workers may use different activities to deal with different tasks, and for the same task, different workers may have different working styles. To discriminate different activities accurately, examples provide a personalized activity recognition method that can automatically create new labeled data to update a personalized activity recognition model.

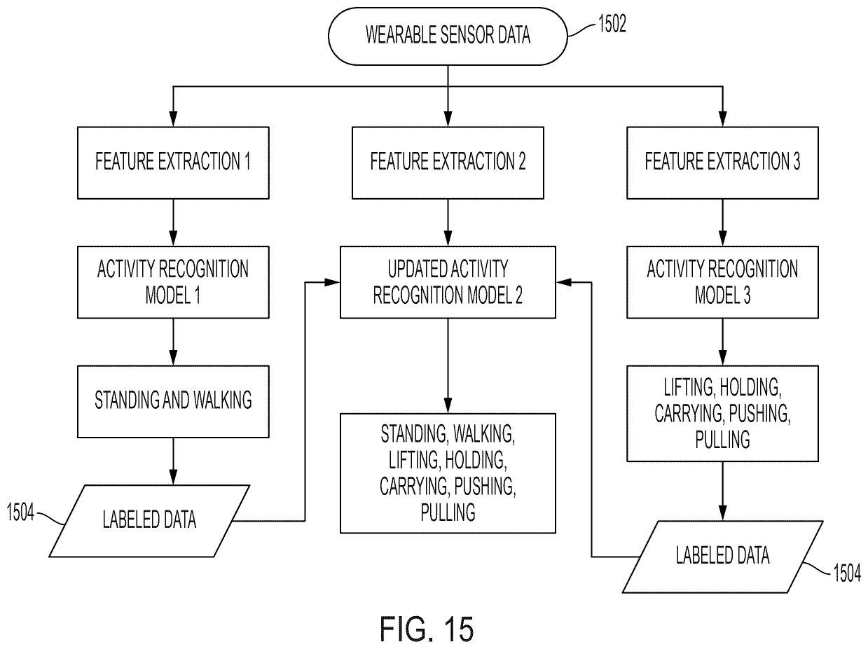

FIG. 15 shows an example of process of personalized lifting activity recognition. Activity recognition may be achieved using sensor data 1502 acquired from the smart wristband 102 and/or smart insole 104.

In one example, the activities are separated into three different groups based on the load weight. In the illustrated example, when load weight≤2 pounds (group 1), the activities can be standing and walking. When load weight≥10 pounds (group 3), the activities can be lifting, holding, carrying, pushing, and pulling. When 2 pounds<load weight<10 pounds (group 2), the activities can be daily living activities and working activities. According to certain examples, standing and walking can be discriminated accurately by a time threshold for foot contact time. Foot contact can be measured using the smart insole 104, as described above. Lifting, pushing, and pulling can be accurately discriminated because they have different plantar pressure distribution patterns, which can also be measured using the smart insole 104 as described above. Further, considering that items being held or carried must be lifted up first, when lifting can be identified accurately, the holding (standing with load) and carrying (walking with load) can also be recognized accurately. To recognize activities in group 2, an example of the methodology assumes that for each activity, the activity patterns in group 1 and group 3 are similar to group 2. For example, the lifting activities for items higher than 10 pounds (e.g. 15 pounds) may be similar to the lifting activities for items lower than 10 pounds (e.g. 5 pounds). Therefore, the activity recognition results in group 1 and group 3 can be used to generate new labeled data 1504 for training the personalized activity recognition model used in group 2. In examples, Support Vector Machine (SVM) and Long Short-Term Memory (LSTM) algorithms are used for recognizing activities in group 1 and group 3, and can be applied to recognize activities in group 2.

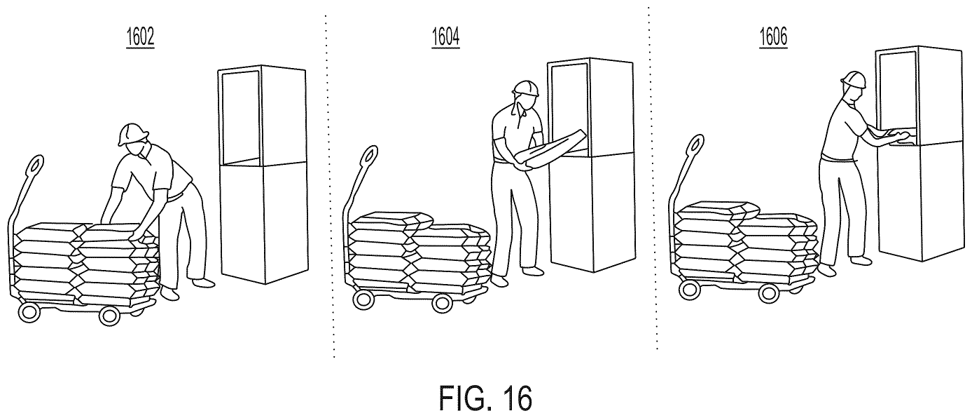

FIG. 16 shows an example of the process of a normal lifting activity. In this example, the worker may wear a smart wristband 102 on each wrist, and a smart insole 104 in both shoes. For manual lifting tasks with both hands, the worker uses both hands to reach to the direction of the load before lifting (e.g., position 1602). Until this point, the angular changes of both hands will be similar and the mean angular changes from the IMUs 202 in the smart wristbands 102 can be used to measure asymmetry angle. Then the hands will grasp and lift the item up (e.g., position 1604). For items that do not have good handles, the posture of two wrists may have significant differences, such that their mean value is not a good indicator of the asymmetry angle. Accordingly, in certain examples, if significant differences in wrist angle changes are detected before lifting, the mean value of similar changes of both wrists may be used to calculate asymmetry angles, and the second zero-asymmetry reference posture (described above) can be relied on for calculation because the second zero-asymmetry reference is not related to the angle of both wrists but related to the changes of the wrist angle during lifting. At a next step in the lifting process (e.g., position 1606), the hands will transfer the load to the direction of the destination, and finally release the hands. The asymmetry angle at the destination can be extracted by using the first or second zero-asymmetry references.

According to certain examples, the start and end of the lifting can be detected by the GRF measured under the feet. Since the hands are involved in the whole lifting process, the load angle can be tracked with the IMUs 202 on the wrists. Therefore, the asymmetry angle may be calculated by tracking the angle of the wrists and feet.

According to certain examples, the vertical travel distance can be estimated with good accuracy by measuring the air pressure changes using calibrated barometers in the smart wristbands 102, as described above. To measure load vertical location, in addition to knowing the vertical travel distance, a reference point for this travel distance is also needed. In some examples, a frequently occurring zero-vertical-location reference posture is used to assist the calculation of the load vertical location relative to the human body. In some examples, the frequently occurring walking activity is used as the zero-reference activity. As discussed above, during walking, arms will swing forward and backward rhythmically. Referring again to FIG. 10, in some examples, zero-reference posture is chosen at the midstance phase during walking, when the supporting foot is flat, the supporting leg is straight, and the swing arm is in the vertical direction beside the thigh. As discussed above, the flexible pressure sensor array 310 and the accelerometer (in the IMUs 202 and 322) can detect the midstance phase and the hand posture reliably. The arm direction can be detected by comparing the gravity direction and the axis of IMU 202. As described above, through updating the air pressure measurements from the barometer sensor 204 using the KVLU reference points, the drift of the barometer(s) can be removed or at least reduced. With the user's height information and the load vertical travel distance measured by the barometer sensor 204, the absolute load location can be estimated with good accuracy. During the same walking cycle, the hand and foot movement directions are along the mid-sagittal plane, and therefore, the asymmetry angle can be updated, as described above.

Thus, aspects and embodiments provide a wearable system for long-term measurement of lifting risk factors includes features and techniques to address the various challenges discussed above. In examples, a force plate is integrated into an insole to supply an absolute reference to automatic calibration of the flexible pressure sensor array. Techniques for automatically calibrating the flexible pressure sensor array, without or without using the wearable force plate, are described above. In addition, barometer sensors in a wristband or glove can be used to measure vertical movement, and can also be automatically calibrated using the techniques described herein. In further examples, techniques are provided for optimizing the measurement of load location and asymmetry angles with personalized activity recognition and zero-reference postures identification.

The following examples pertain to further embodiments, from which numerous permutations and configurations will be apparent.

Example 1 is a wearable sensor system for lifted load measurements, the system comprising: an auto-calibrating insole apparatus including an insole, a flexible pressure sensor array coupled to the insole and including a plurality of pressure sensors distributed over a surface area of the insole, a force plate integrated into a heel region of the insole, and a first inertial measurement unit (IMU) coupled to the insole; and at least one sensor module configured to be worn on one or both wrists or hands of a user.

Example 2 includes the wearable sensor system of Example 1, wherein the force plate includes a plurality of micro load cells.

Example 3 includes the wearable sensor system of one of Examples 1 or 2, wherein the force plate is configured to provide a reference force measurement for automatic calibration of the flexible pressure sensor array.

Example 4 includes the wearable sensor system of any one of Examples 1-3, wherein the at least one sensor module includes a barometer, and a second IMU.

Example 5 is a wearable sensor system for lifted load measurements, the system comprising: an auto-calibrating first sensor system including an insole, a flexible pressure sensor array coupled to the insole and including a plurality of pressure sensors distributed over a surface area of the insole, and a first inertial measurement unit (IMU); an auto-calibrating second sensor system configured to be worn on a wrist or hand of a user, the auto-calibrating second sensor system including a second IMU and at least one barometer; and at least one processor coupled to the first and second sensor systems and configured to implement a first automatic calibration procedure for the flexible pressure sensor array based on first sensor data acquired using the flexible pressure sensor array and the first IMU, the at least one processor being further configured to implement a second automatic calibration procedure for the at least one barometer based on the first sensor data and on second sensor data acquired using the second IMU.

Example 6 includes the wearable sensor system of Example 5, wherein the auto-calibrating second sensor system is configured to measure load vertical location based on air pressure measurements acquired using the at least one barometer.

Example 7 includes the wearable sensor system of Example 6, wherein to implement the second automatic calibration procedure, the at least one processor is configured to identify at least one reference point having a determined vertical location.

Example 8 includes the wearable sensor system of Example 7, wherein the determined vertical location is estimated based on a body height of the user and a wrist-to-height ratio.

Example 9 includes the wearable sensor system of one of Examples 7 or 8, wherein the at least one processor is configured to recognize a walking or standing activity based on the first sensor data, and to identify the at least one reference point during the walking or standing activity.

Example 10 includes the wearable sensor system of any one of Examples 5-9, wherein the auto-calibrating second sensor system is a first wristband system configured to be worn on a first wrist of the user; wherein the wearable sensor system further comprises a second wristband system configured to be worn on a second wrist of the user, the second wristband system being substantially identical to the first wristband system; and wherein the wearable sensor system is configured to determine an asymmetry angle during load lifting based on the second sensor data from the first and second wristband systems.

Example 11 includes the wearable sensor system of any one of Examples 5-10, wherein to implement the first automatic calibration procedure, the at least one processor is configured to: apply a machine learning model using at least a portion of the first sensor data to produce an estimated normalized ground reaction force (GRF) pattern for a whole foot region corresponding to the insole, convert the estimated normalized GRF pattern to units of force to produce an estimated absolute whole foot GRF pattern, determine an average regression curve based on the absolute whole foot GRF pattern using least squares estimation, based on the average regression curve, estimate GRF values for individual pressure sensors of the flexible pressure sensor array, adjust the estimated GRF values based on based on foot geometry information to produce regression curves for the individual pressure sensors, and calibrate the force measurements from the individual sensors based on the regression curves.

Example 12 includes the wearable sensor system of Example 11, wherein the at least one processor is configured to convert the estimated normalized GRF pattern to units of force based on a body weight of the user.

Example 13 includes the wearable sensor system of any one of Examples 5-12, wherein the first sensor system further includes a force plate integrated into a heel region of the insole and configured to provide reference force measurements.

Example 14 includes the wearable sensor system of Example 13, wherein the force plate includes a plurality of micro load cells.

Example 15 includes the wearable sensor system of Example 14, wherein the plurality of micro load cells includes four micro load cells, one positioned at each corner of the force plate.

Example 16 includes the wearable sensor system of any one of Examples 5-15, wherein the at least one processor includes a microcontroller coupled to the insole.

Example 17 includes the wearable sensor system of any one of Examples 5-15, further comprising a computing device that comprises the at least one processor.

Example 18 includes the wearable sensor system of Example 17, wherein the computing device is a smartphone or tablet.

Example 19 includes the wearable sensor system of any one of Examples 5-18, wherein the first IMU is coupled to the insole.

Example 20 includes the wearable sensor system of any one of Examples 5-19, wherein the first sensor system and the second sensor system each comprises a wireless communications interface.

Example 21 includes the wearable sensor system of Example 20, wherein the wireless communications interface is a BLUETOOTH interface.

Example 22 includes a monitoring system comprising: at least one first sensor system including an insole, a flexible pressure sensor array coupled to the insole and configured to provide pressure sensor data, a first inertial measurement unit (IMU) configured to provide first IMU data, and a first wireless communication interface, wherein the flexible pressure sensor array includes a plurality of pressure sensors distributed over a surface area of the insole; at least one second sensor system configured to be worn on a wrist or hand of a user, the at least one second sensor system including a second IMU configured to provide second IMU data, at least one barometer configured to provide air pressure measurements, and a second wireless communications interface; and a computing device configured to receive the pressure sensor data and the first IMU data from the at least one first sensor system via the first wireless communication interface and configured to receive the second IMU data and the air pressure measurements from the at least one second sensor system via the second wireless communications interface, the computing device further configured to calibrate the at least one barometer based on the pressure sensor data and the second IMU data, and to calibrate the flexible pressure sensor array based on the first IMU data.

Example 23 includes the monitoring system of Example 22, wherein the at least one first sensor system further comprises a force plate integrated into a heel region of the insole and configured to provide reference force measurements; wherein the computing device is configured to receive the reference force measurements from the force plate via the first wireless communications interface; and wherein the computing device is configured to calibrate the flexible pressure sensor array based on the first IMU data and the reference force measurements.

Example 24 includes the monitoring system of Example 23, wherein the force plate includes a plurality of micro load cells.

Example 25 includes the monitoring system of any one of Examples 22-24, wherein the first wireless communications interface and the second wireless communications interface are BLUETOOTH interfaces.

Example 26 includes the monitoring system of any one of Examples 22-26, wherein the at least one first sensor system includes a pair of first sensor systems configured to be worn in left and right shoes of the user, and wherein the at least one second sensor system includes a pair of second sensor systems configured to be worn on left and right wrists of the user.

Example 27 includes the monitoring system of any one of Examples 22-26, wherein the at least one second sensor system is configured to measure load vertical location based on the air pressure measurements from the at least one barometer.

Having described above several aspects of at least one embodiment, it is to be appreciated various alterations, modifications, and improvements will readily occur to those skilled in the art. Such alterations, modifications, and improvements are intended to be part of this disclosure and are intended to be within the scope of the invention. Accordingly, the foregoing description and drawings of various embodiments are presented by way of example only. These examples are not intended to be exhaustive or to limit the invention to the precise forms disclosed. The methods and apparatuses are capable of implementation in other embodiments and of being practiced or of being carried out in various ways. In addition, the phraseology and terminology used herein is for the purpose of description and should not be regarded as limiting. Any references to examples, components, elements, or acts of the systems and methods herein referred to in the singular can also embrace examples including a plurality, and any references in plural to any example, component, element or act herein can also embrace examples including only a singularity. References in the singular or plural form are not intended to limit the presently disclosed systems or methods, their components, acts, or elements. The use herein of “including”, “comprising”, “having”, “containing”, “involving”, and variations thereof is meant to encompass the items listed thereafter and equivalents thereof as well as additional items. References to “or” can be construed as inclusive so that any terms described using “or” can indicate any of a single, more than one, and all of the described terms.

1. A wearable sensor system for lifted load measurements, the system comprising:
an auto-calibrating first sensor system including an insole, a flexible pressure sensor array coupled to the insole and including a plurality of pressure sensors distributed over a surface area of the insole, and a first inertial measurement unit (IMU);
an auto-calibrating second sensor system configured to be worn on a wrist or hand of a user, the auto-calibrating second sensor system including a second IMU and at least one barometer, wherein the auto-calibrating second sensor system is configured to measure load vertical location based on air pressure measurements acquired using the at least one barometer; and
at least one processor coupled to the first and second sensor systems and configured to implement a first automatic calibration procedure for the flexible pressure sensor array based on first sensor data acquired using the flexible pressure sensor array and the first IMU, the at least one processor being further configured to implement a second automatic calibration procedure for the at least one barometer based on the first sensor data and on second sensor data acquired using the second IMU;
wherein to implement the second automatic calibration procedure, the at least one processor is configured to identify at least one reference point having a determined vertical location; and
wherein the determined vertical location is estimated based on a body height of the user and a wrist-to-height ratio.

2. (canceled)

3. (canceled)

4. (canceled)

5. The wearable sensor system of claim 4, wherein the at least one processor is configured to:
recognize a walking or standing activity based on the first sensor data; and
identify the at least one reference point during the walking or standing activity.

6. The wearable sensor system of claim 1, wherein the auto-calibrating second sensor system is a first wristband system configured to be worn on a first wrist of the user
wherein the wearable sensor system further comprises a second wristband system configured to be worn on a second wrist of the user, the second wristband system being substantially identical to the first wristband system; and
wherein the wearable sensor system is configured to determine an asymmetry angle during load lifting based on the second sensor data from the first and second wristband systems.

7. The wearable sensor system of claim 1, wherein to implement the first automatic calibration procedure, the at least one processor is configured to:
apply a machine learning model using at least a portion of the first sensor data to produce an estimated normalized ground reaction force (GRF) pattern for a whole foot region corresponding to the insole,
convert the estimated normalized GRF pattern to units of force to produce an estimated absolute whole foot GRF pattern,
determine an average regression curve based on the absolute whole foot GRF pattern using least squares estimation,
based on the average regression curve, estimate GRF values for individual pressure sensors of the flexible pressure sensor array,
adjust the estimated GRF values based on based on foot geometry information to produce regression curves for the individual pressure sensors, and
calibrate the force measurements from the individual sensors based on the regression curves.

8. The wearable sensor system of claim 7, wherein the at least one processor is configured to convert the estimated normalized GRF pattern to units of force based on a body weight of the user.

9. The wearable sensor system of claim 1, wherein the first sensor system further includes a force plate integrated into a heel region of the insole and configured to provide reference force measurements.

10. The wearable sensor system of claim 9, wherein the force plate includes a plurality of micro load cells.

11. The wearable sensor system of claim 1, wherein the at least one processor includes a microcontroller coupled to the insole.

12. The wearable sensor system of claim 1, further comprising a computing device that comprises the at least one processor.

13. The wearable sensor system of claim 12, wherein the computing device is a smartphone or tablet.

14. The wearable sensor system of claim 1, wherein the first IMU is coupled to the insole.

15. The wearable sensor system of claim 1, wherein the first sensor system and the second sensor system each comprises a wireless communications interface.

16. A monitoring system comprising:
at least one first sensor system including an insole, a flexible pressure sensor array coupled to the insole and configured to provide pressure sensor data, a first inertial measurement unit (IMU) configured to provide first IMU data, and a first wireless communication interface, wherein the flexible pressure sensor array includes a plurality of pressure sensors distributed over a surface area of the insole;
at least one second sensor system configured to be worn on a wrist or hand of a user, the at least one second sensor system including a second IMU configured to provide second IMU data, at least one barometer configured to provide air pressure measurements, and a second wireless communications interface, wherein the at least one second sensor system is configured to measure load vertical location based on the air pressure measurements acquired using the at least one barometer; and
a computing device configured to receive the pressure sensor data and the first IMU data from the at least one first sensor system via the first wireless communication interface and configured to receive the second IMU data and the air pressure measurements from the at least one second sensor system via the second wireless communications interface, the computing device further configured to calibrate the at least one barometer based on the pressure sensor data and the second IMU data, and to calibrate the flexible pressure sensor array based on the first IMU data;
wherein to calibrate the at least one barometer, the computing device is configured to identify at least one reference point having a determined vertical location; and
wherein the determined vertical location is estimated based on a body height of the user and a wrist-to-height ratio.

17. The monitoring system of claim 16, wherein the at least one first sensor system further comprises a force plate integrated into a heel region of the insole and configured to provide reference force measurements;
wherein the computing device is configured to receive the reference force measurements from the force plate via the first wireless communications interface; and
wherein the computing device is configured to calibrate the flexible pressure sensor array based on the first IMU data and the reference force measurements.

18. The monitoring system of claim 17, wherein the first wireless communications interface and the second wireless communications interface are BLUETOOTH interfaces.

19. The monitoring system of claim 16, wherein the at least one first sensor system includes a pair of first sensor systems configured to be worn in left and right shoes of the user; and
wherein the at least one second sensor system includes a pair of second sensor systems configured to be worn on left and right wrists of the user.

20. The monitoring system of claim 19, wherein the computing device is configured to determine an asymmetry angle during load lifting based on the second IMU data from the pair of second sensor systems.

21. The monitoring system of claim 16, wherein the computing device is further configured to:
recognize a walking or standing activity based on first sensor data acquired using the flexible pressure sensor array; and
identify the at least one reference point during the walking or standing activity.

22. The monitoring system of claim 17, wherein the force plate includes a plurality of micro load cells.
